(19) United States
(12) Patent Application Publication
(10) Pub. No.: US 2026/0295539 A1
(43) Pub. Date: Oct. 1, 2026

(54) IN LINE CLEANING IN PLACE PROCESS FOR ALTERNATING TANGENTIAL FLOW FILTER

(71) Applicant: ENZENE BIOSCIENCES LIMITED, Pune, Maharashtra (IN)

(72) Inventors: Mahesh R. REDDY, Pune (IN); Vijaya Lakshmi VAMANAN, Pune (IN); Shreya GHOSH, Pune (IN); Kunal BARDIYA, Pune (IN); Veerendra Kumar P. REDDY, Pune (IN); Shilpa GADGIL, Pune (IN); Himanshu GADGIL, Pune (IN)

(73) Assignee: ENZENE BIOSCIENCES LIMITED, Pune, Maharashtra (IN)

(21) Appl. No.: 19/478,728

(22) PCT Filed: Apr. 25, 2024
(86) PCT No.: PCT/IB2024/054052

§ 371 Date: Oct. 24, 2025

(30) Foreign Application Priority Data
Apr. 25, 2023 (IN) 202321029898

(51) Int. Cl. B01D 65/06; C12M 1/00

(52) U.S. Cl. CPC B01D 65/06; C12M 39/00; B01D 2315/10; B01D 2321/02; B01D 2321/164

(57) ABSTRACT

The present invention provides a process for an in line cleaning in place (CIP) of an alternating tangential flow (ATF) filter in a filtration module, the filtration module being in communication with a bioreactor. The process comprises: flowing a dilute NaOH through the ATF filter, the dilute NaOH being 0.1 to 0.5 N NaOH, the volume of NaOH being three to five times the hold up volume of the filter and the flow rate of NaOH being 80-200 ml/L; flushing the filter with a sterile water, the volume of water being flushed being ten to twenty times the hold up volume of the filter; and equilibrating the filter with a media. The present invention also provides a system for an in line CIP of an alternating tangential flow (ATF) filter in a filtration module, the filtration module being in communication with a bioreactor.

### FIELD OF THE INVENTION

The present disclosure relates to an in line cleaning in place (CIP) process for alternating tangential flow (ATF) filters. Particularly, the present disclosure relates to a process for an in line cleaning in place (CIP) of an alternating tangential flow (ATF) filter in a filtration module, which is in communication with a bioreactor to alleviate protein retention in the bioreactor. In particular, the present disclosure provides a process of an in line CIP to remove cell debris or blockages retained in or on the filter and help reuse the ATF filter for a longer period for the same batch.

### BACKGROUND OF THE INVENTION

Cell cultures have been widely applied to produce biologics like proteins, receptors, vaccines and antibodies for and array of applications; for example as therapeutics, for research and diagnostics. Various cell culture techniques have been developed to address challenges such as low productivity, contamination, high costs etc.

Perfusion cell culture has attracted interest as it allows cells to stay in the exponential growth phase for an extended time and reach higher viable cell densities. However, substantial quantity of debris and cell waste gets accumulated inside the bioreactor; during, the production as a result of the high density of the cell lines utilized in the mammalian cell process, which causes protein retention inside the bioreactor, reducing the yield of the harvested protein. To address such limitations, in tandem, an alternating tangential flow (ATF) systems using hollow fiber filters have been developed for perfusion processes employing prokaryotic and eukaryotic cell cultures, to achieve efficient cell separation. ATF filters have been used to remove cell-free harvest.

In the ATF system, a cell retention device keeps the cells within the bioreactor; while, the spent media-containing product is harvested. The cells are then pushed back into the bioreactor by the alternating flow. Thus, ATF systems have considered to be separation system of choice. However, employing ATF filters also suffers from drawbacks; especially, with single-use products, where a new ATF filter needs to be changed each time the filter clogs, which will halts the production process and raises the expense of production.

Even though, it has been reported that Alternating Tangential Flow (ATF) system has less protein retention than other tangential flow filtration (TFF) based perfusion systems. Protein retention has been observed for the majority of the molecules. Various levels of protein retention are frequently observed in the perfusion process with different CHO cell lines; i.e. GS (glutamine synthetase), DHFR (dihydrofolate reductase), and CHOK1, or the like. Protein retention in perfusion culture can be caused by several factors, including antifoam, cell debris with different particle sizes, medium components, and protein-related problems, either separately or in combination. The influence the back-and-forth media flow on cell health and protein quality during the ATF based process is not well understood; but, it is found to increase the level of shear stress experienced by the cells and protein products. Mammalian cells are particularly sensitive to shear or mechanical stress and various studies have shown that high levels of shear can affect the cell viability and growth. This has necessitated designing the process with operation parameters carefully that does not disturb the hydrodynamic conditions significantly to avoid damage to the cells and protein productions.

Various previous attempts include an approach as per the published PCT patent application WO2005097306A1, wherein it discloses cleaning membrane filters comprising hollow fibres and an inner skin. The method disclosed therein comprises the following steps consisting in: emptying the concentrate compartment in order to release the liquid to be filtered contained therein and the suspended matter, and, subsequently, performing a backwashing step involving the passage of liquid from the permeate compartment into the concentrate compartment through membranes in order to detach and release impurities deposited thereon, while circulating a gas in the concentrate. According to the invention, backwashing liquid and/or gas pulses are produced by control means (5, 5a; 7, 7a) during at least one backwashing phase, which can subject the cells and protein product and filter membrane to undesired stress, which can have adverse implication on the cells, protein product and make the membrane weak or prone damage and inefficient.

Another approach includes as disclosed in the published PCT application WO2005028085A1, which discloses a method and apparatus for backwashing a membrane filtration system wherein permeate remaining present in the filtration system, when the filtration process is stopped or suspended is used to provide liquid for backwashing the membrane pores during a backwashing process. Such system is required to stop the filtration system; which can delay the over all product, render the process inefficient and add to the cost of the product.

Currently, the so-called cleaning in place systems and processes known as CIP have been considered for cleaning bioreactors, filtration units and allied equipment without dismantling. However, most CIP processes involve use of multiple harsh reagents such as detergents, disinfectants, alkalis, surfactants, acids at higher concentrations and temperatures, that can leave harmful traces inside the system and renders them unsuitable for applying them in an in line mode. Most often, the use of harsh alkaline solution, and acids dissolves proteins and thereby making such processes suitable for protein production set-ups, including ATF filtration units.

Thus, there remains an unmet need to provide an in line cleaning in process that can be specifically adopted for ATF filtration systems including single-use ATF filters, which can be cleaned effectively by removing the cell debris, residues and left over materials retained in and over the filters to circumvent the need to change the filler each time the filter clogs, avoid halting the production process, allowing the reuse of the filter and importantly that can address the challenge of retention or accumulation of protein product inside the bioreactor.

### OBJECTS OF THE INVENTION

An object of the present disclosure is to provide a process for an in line cleaning in place (CIP) of alternating tangential flow (ATF) filters.

Another object of the present disclosure is to provide a process for an in line cleaning in place (CIP) of alternating tangential flow (ATF) filters allowing their reusability.

Yet another object of the present disclosure is to provide a process for an in line cleaning in place (CIP) of alternating tangential flow (ATF) filters to address the challenge with protein retention in the bioreactor.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

In a general aspects of the present disclosure provide an in line cleaning in place (CIP) for alternating tangential flow (ATF) filters.

Aspects of the present disclosure provide an in line cleaning in place (CIP) for alternating tangential flow (ATF) filters that can overcome one or more of the disadvantages existing in the art.

The present disclosure provides a process for an in line CIP of filter to help remove cell debris or blockages inside the filters and help reuse the ATF filter for a longer period for the same batch.

This straightforward in line CIP is made for industrial-scale manufacturing bioreactors that use different types of ATF 2, 4, 6, and 10.

Another aspect of the present disclosure is to provide a process that can allow reusability of ATF hollow fiber filter by implementing CIP.

Yet another aspect of the present disclosure is to provide an improved process to address protein retention challenges.

In one aspect, the present disclosure provides a process for an in line cleaning in place (CIP) of an alternating tangential flow (ATF) filter in a filtration module, the filtration module being in communication with a bioreactor, the process comprising the steps of:
i) flowing a dilute NaOH through the ATF filter, the dilute NaOH being 0.1 to 0.5 N NaOH, the volume of NaOH being three to five times the hold up volume of the filter and the flow rate of NaOH being 80-200 ml/L;
ii) flushing the filter with a sterile water, the volume of water being flushed being ten to twenty times the hold up volume of the filter; and
iii) equilibrating the filter with a media.

The present invention also provides a system for an in line CIP of an alternating tangential flow (ATF) filter in a filtration module, the filtration module being in communication with a bioreactor.

The present invention overcome the protein retention issue and allow for the lengthy batch duration without frequent changes of the ATF hollow fiber filter.

Other aspects of the invention will be set forth in the description which follows, and in part will be apparent from the description, or may be learnt by the practice of the invention.

### BRIEF DESCRIPTION OF THE DRAWINGS

FIG. 1: illustrates schematic representation of an experimental system set up in accordance with the present invention comprising bioreactor connected with an ATF filtration module and the in line CIP process implemented with the said set up.

FIG. 2: illustrates growth profile of Denosumab in a Perfusion Batch.

FIG. 3: illustrates productivity profile of Denosumab Perfusion Harvest with in line CIP of the present invention implemented.

FIG. 4: illustrates growth profile of Cetuximab perfusion batch.

FIG. 5: illustrates productivity profile for Cetuximab Perfusion Harvest with in line CIP of the present invention implemented. The bioreactor titre, which accumulated over the batch due to ATF filter choking, is displayed by the upper graph and the harvest is represented by the lower graph, and the peak harvest titre was observed 0.7 g/L/Day following multiple CIP.

FIG. 6: illustrates growth profile of Ravulizumab—I in a Perfusion Batch. The inoculation VCC was 5 million cells per mL, and peak VCC was observed 87.6 million cells per mL on Day 6. IN CIP was done on Days 10 and 14.

FIG. 7: illustrates productivity profile of Ravulizumab—I Perfusion Harvest titre with in line CIP of the present invention implemented.

FIG. 8: illustrates productivity profile of Ravulizumab—I Perfusion Bioreactor titre with in line CIP of the present invention implemented. The bioreactor titre of 1.3 g/L, which was accumulated over the batch due to ATF filter choking. The peak harvest titre was observed 0.6 g/L/Day following multiple CIP,

FIG. 9: illustrates growth profile of Ravulizumab—II in a Perfusion Batch. The inoculation VCC was 0.5 million cells per mL, and peak VCC was observed 805 million cells per mL on Day 14. IN CIP was done on Days 10.

FIG. 10: illustrates productivity profile of Ravulizumab—II Perfusion Harvest with in line CIP of the present invention implemented.

FIG. 11: illustrates productivity profile of Ravulizumab—II Perfusion Bioreactor titre with in line CIP of the present invention implemented. The bioreactor titre of 5 g/L was the result of an ATF filter choking, which accumulated over the batch. After several CIPs, the peak harvest titre was recorded at 0.6 g/L/Day.

FIG. 12: illustrates growth profile of Tocilizumab in a Perfusion Batch. The inoculation VCC was 0.5 million cells per mL, and peak VCC was observed 90 million cells per mL on Day 10. IN CIP was done on Days 15

FIG. 13: illustrates productivity profile of Tocilizumab Perfusion Harvest with in line CIP of the present invention implemented.

FIG. 14: illustrates productivity profile of Tocilizumab Perfusion Bioreactor titre with in line CIP of the present invention implemented. The bioreactor titre of 1.1 g/L was the result of an ATF filter choking, which accumulated over the batch. After CIP, the peak harvest titre was recorded at 0.8 g/L/Day.

FIG. 15: illustrates growth profile of Nivolumab in a Perfusion Batch. The inoculation VCC was 0.5 million cells per mL, and peak VCC was observed 80 million cells per mL and IN CIP was done on Day 15.

FIG. 16: illustrates productivity profile of Nivolumab Perfusion Harvest with in line CIP of the present invention implemented.

FIG. 17: illustrates productivity profile of Nivolumab Perfusion Bioreactor titre with in line CIP of the present invention implemented. The bioreactor titre of 1.5 g/L was the result of an ATF filter choking, which accumulated over the batch and the CIP was performed on day 15.

FIG. 18: illustrates growth profile of Pembrolizumab in a Perfusion Batch. The inoculation VCC was 0.5 million cells per mL, and peak VCC was observed 70 million cells per mL on Day 17. IN CIP was done on Days 22, 25 and 26.

FIG. 19: illustrates productivity profile of Pembrolizumab Perfusion Harvest with in line CIP of the present invention implemented.

FIG. 20: illustrates productivity profile of Pembrolizumab Perfusion Bioreactor titre with in line CIP of the present invention implemented. The bioreactor titre of 4.3 g/L was the result of an ATF filter choking, which accumulated over the batch. After several CIPs, the peak harvest titre was recorded at 2.5 g/L/Day.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the scope of the present disclosure.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weights, percentages, ratios, and so forth, to describe certain embodiments of the invention and are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Various terms as used herein are shown below. To the extent a term used is not defined below, it should be given the broadest definition persons in the pertinent art have given that term as reflected in printed publications and issued patents at the time of filing.

As used in the description herein that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention.

Groupings of alternative elements or embodiments of the invention disclosed herein are not to be construed as limitations. Each group member can be referred to individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used herein the term Hold up volume is volume retained in the filter or used in the bioreactor.

Lag phase refers to the phase of cell cycle; wherein cells do not divide. It is the period when cells are adjusting to the culture condition and preparing for the cell division. In our invention, first 2-5 days are considered as lag phase.

Log phase, also known as logarithmic phase or exponential phase. It refers to the phase of cell cycle, when cells actively proliferate and the cell density increases exponentially. It is recommended to assess cellular function at this stage since the cell population is most viable. In our invention, first 5-12 days are considered as log Phase.

Stationary phase (or plateau phase) refers to cell cycle phase; wherein cell proliferation slows down due to a growth-limiting factor such as the depletion of an essential nutrient and/or the formation of an inhibitory product, resulting in a situation in which growth rate and death rate are equal. In our invention, day 12-30 are considered as Stationary phase.

Decline phase refers to cell cycle phase wherein cell death predominates and the number of viable cells reduces. In our invention, last 2-4 days of growth cycle is considered as decline phase.

ATF as used herein is an acronym of alternating tangential flow and it uses hollow porous fibrous membranes (pore sizes such as 0.1-5.0 micron or ultrafiltration membranes such as 750 kDa MWCO) to retain the cells and other particulate matter. ATF system includes a bioreactor for harvest, particularly a stirred tank reactor. Bioreactor is connected with ATF via a tubes to maintain the sterile conditions. ATF system perfuse a harvested cell culture in the bioreactor using hollow porous fibrous membranes and alternating tangential flow Moreover, ATF system also includes a controller to control the diaphragm pump which perform the ATF using hollow porous fibrous membranes.

The present disclosure is directed to address various shortcoming existing in the art such as protein retention in the bioreactor and interruption of the process for changing or cleaning the filter.

In an embodiment, the present disclosure discloses a process for an in line cleaning in place (CIP) of an alternating tangential flow (ATF) filters.

In an embodiment, the present disclosure discloses a process for an in line cleaning in place (CIP) of an alternating tangential flow (ATF) filters with 0.1 to 0.5 N sodium hydroxide.

The present disclosure provides a process allowing reusability of ATF hollow fiber by implementing CIP.

The present disclosure provides an improved process that addresses challenges of protein retention in the bioreactor and associated disadvantages.

In an embodiment, the present invention provides a process for an in line cleaning in place (CIP) of an alternating tangential flow (ATF) filter in a filtration module, the filtration module being in communication with a bioreactor, the process comprising the steps of:
i) flowing a dilute NaOH through the ATF filter, the dilute NaOH being 0.1 to 0.5 N NaOH, the volume of NaOH being three to five times the hold up volume of the filter and the flow rate of NaOH being 80-200 ml/L;
ii) flushing the filter with a sterile water, the volume of water being flushed being ten to twenty times the hold up volume of the filter; and
iii) equilibrating the filter with a media.

In an embodiment of the present disclosure the process includes monitoring a titer of a product in the bioreactor.

In an embodiment of the present disclosure the in line CIP is initiated based on the titre value of the accumulated product in the bioreactor.

In an embodiment of the present disclosure the in line CIP is carried out one or more times based on of titre value of the accumulated product during a phase selected from a log phase, stationary phase and decline phase of growth of cells cultured in the bioreactor.

In an embodiment of the present disclosure the in line CIP is carried out based on the titre value of the accumulated product in the bioreactor is >20% during the lag phase, >40% during the log phase, >60% during the stationary phase, or <40% and equal to 20% during the decline phase of growth of cells cultured in the bioreactor.

In an embodiment of the present disclosure the in line CIP is repeated one or more times based on the titre value of the accumulated product in the bioreactor is 60% to 70%.

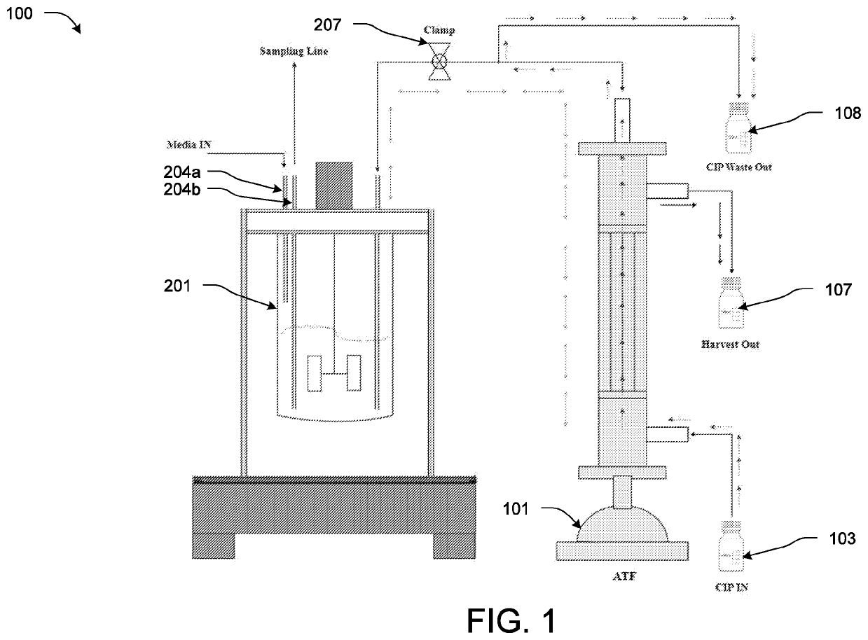

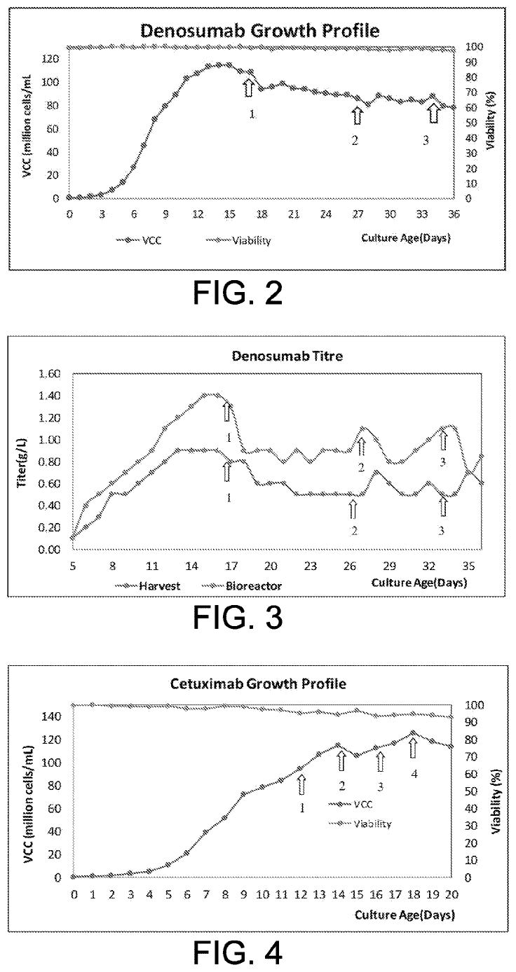

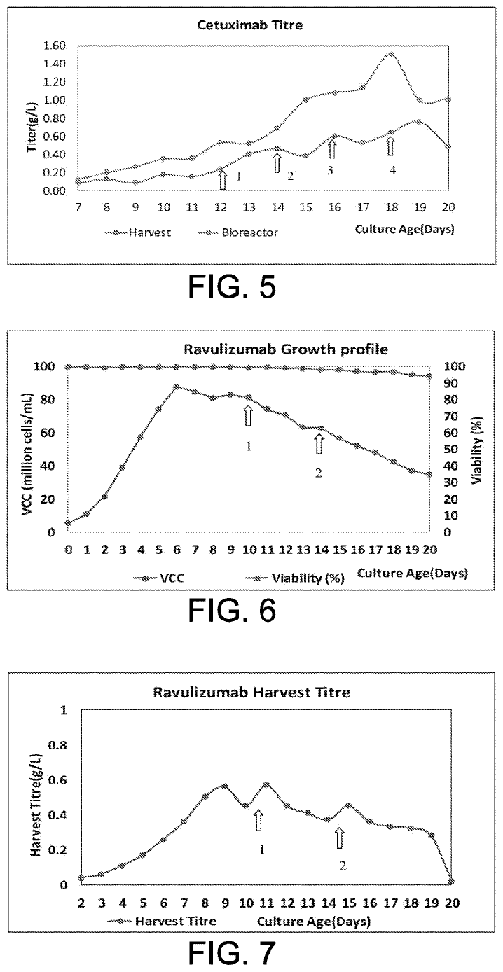

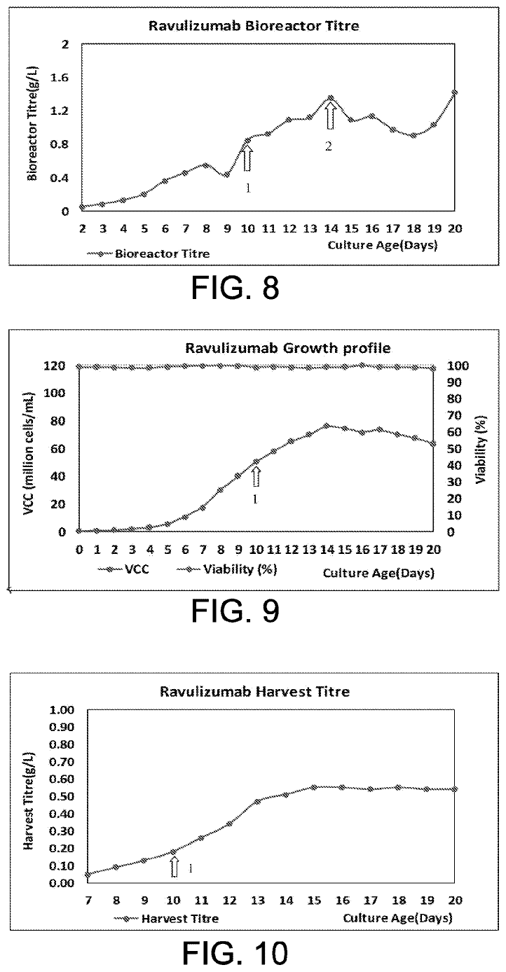

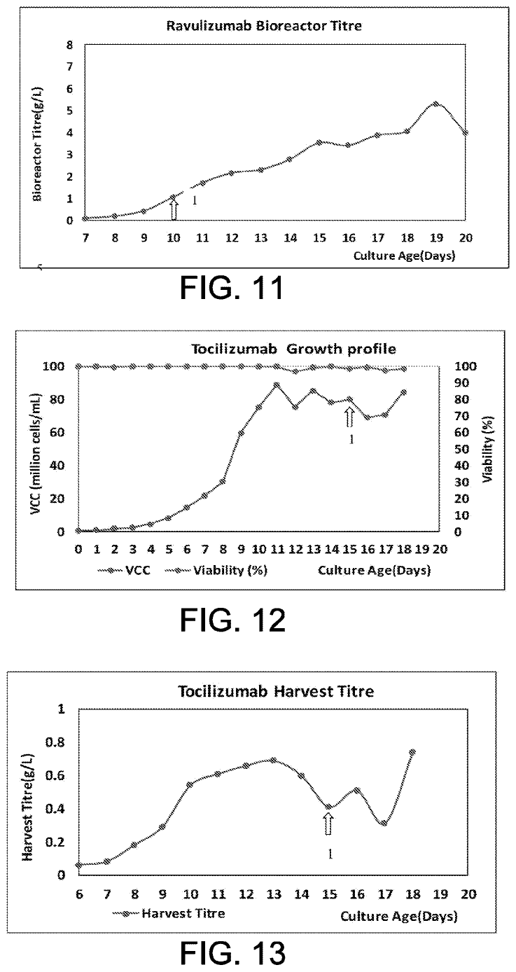

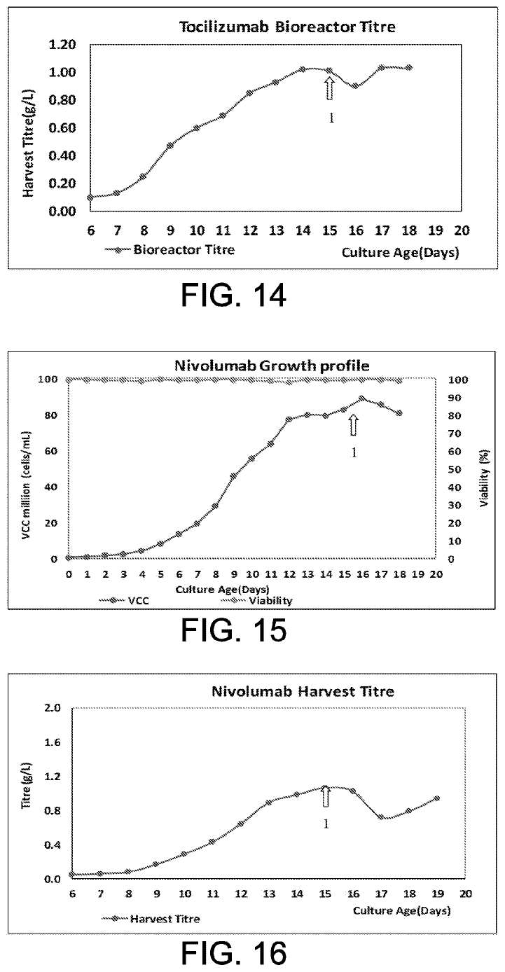

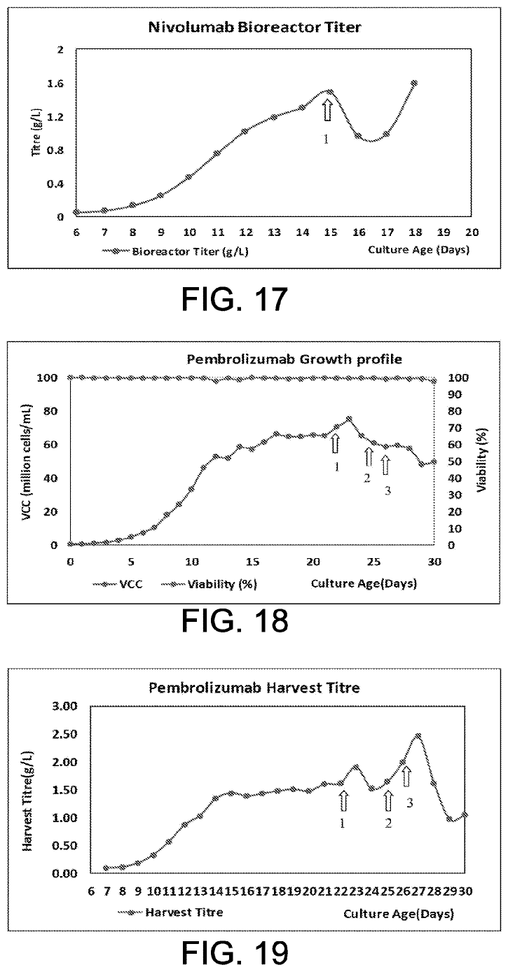

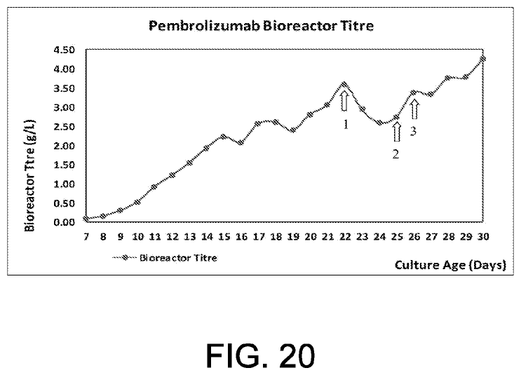

Referring to FIG. 1 it depicts an experimental set-up to carry the process in accordance with the present invention for an in line cleaning in place (CIP) of an alternating tangential flow (ATF) filter, wherein the system (100) comprises: (a) an alternating tangential flow (ATF) filtration module (101) and (b) a bioreactor (201), the ATF filtration module (101) being in communication with a bioreactor (201). The bioreactor (201) includes a cell culture vessel (201a) housed in outer jacket (201b). The cell culture vessel contains a cell culture medium with cells cultured in the same (202), a stirring means (203), a port for introducing the culture medium (204a), a port for sample withdrawal (204b), and port (205) for connecting the bioreactor via a conduit (206) and through clamp (207) with and to be in communication with the ATF filtration module (101) thought port (106). The port (204a) or additional ports (not shown) of the cell culture vessel allows for introducing buffers, air and other required input materials into the cell culture vessel (201a). The flow of air/gases to be introduced into the cell culture vessel is controlled though a mass flow controller (MFC) (not shown) connected to the bioreactor. The conduit (206) allows flow of spent medium containing cells and product from the cell culture vessel (201a) of the bioreactor (201) to the ATF filtration module (101), and flow of liquid medium with separated cells from the ATF filtration module (101) back into the cell culture vessel (201a). The ATF filtration module comprises an hollow fiber filter (102), an inlet port (104) allowing introducing dilute NaOH from vessel (103) and flow through the hollow fiber filter (102) housed within the filtration module (101). The inlet port (104) also allows for introducing sterile water for flushing the hollow fiber filter (102). An outlet port (105) allows withdrawing the harvest from the ATF module (101) containing the product to be collected in a vessel (107). The port (106), through the conduit (206) allows discharge of NaOH and water after flowing through the filter (102), which is collected in a vessel (108).

FIG. 1 further depicts an outline of the process in accordance with the present invention for an in line cleaning in place (CIP) of an alternating tangential flow (ATF) filter in a filtration module (101), the filtration module (101) being in communication with a bioreactor (201), the process comprising the steps of: flowing a dilute NaOH from vessel (103) via inlet port (104) through a hollow fiber ATF filter (102) contained within the filtration module (101), the dilute NaOH being 0.1 to 0.5 N NaOH, the volume of NaOH being three to five times the hold up volume of the filter (102) and the flow rate of NaOH being 80-200 ml/L; flushing the filter (101) with a sterile water via inlet port (104), the volume of water being flushed being ten to twenty times the hold up volume of the filter (102); and equilibrating the filter via inlet port (104) with a media.

In FIG. 1 double arrow represent flow of medium containing cells and product from the bioreactor to the ATF filtration module and separated cells with medium from the ATF filtration module to the bioreactor. Single arrows show the flow of liquid from the one component to the other component as shown.

The process of the present invention can be easily implemented with ATF modules of various capacity for example:

| | | |
|---|---|---|
| ATF-2 | 500-1000 | mL |
| ATF-4 | 3.85-10 | L |
| ATF-6 | 12.5-20 | L |
| ATF-10 | 50-75 | L |

The initiation or starting point for inline CIP can depend on the phase of the cell growth in production culture system which divides into four phases-lag, log, stationary and decline phase. In the lag phase, inline CIP initiated when the titre is in the range of 10-30% inside the bioreactor; while, in the log phase, inline CIP is initiated when the titre is in the range of 30-50% inside the bioreactor. Moreover, in the stationary phase of the cell growth, inline CIP is started at titre difference in the range of 50-80% compared with inside the bioreactor and harvest. In the decline phase, inline CIP is initiated when the titre is 10-30% inside the bioreactor.

| Sr. No. | Cell Growth Phase | Inline CIP Initiation Point |
|---|---|---|
| 1 | Lag Phase | 10-30% |
| 2 | Log Phase | 30-50% |
| 3 | Stationary Phase | 50-80% |
| 4 | Decline Phase | 10-30% |

After CIP, the culture loses about 2 to 10% of its volume, depending on the size of the bioreactor, resulting in a slight loss of cells and dilution of the culture. However, in contrast to bioreactor productivity, harvest productivity has surprisingly found to increase.

The problem of the existing arts has been solved by the present disclosure. The pores of hollow fibers that are partially or completely clogged are cleaned by the in line CIP process of the present invention, which also addresses the problem of protein retention. In line CIP removes cell debris of different sizes, blockages, or layers formed inside the hollow fibre filters by cell lysis, antifoam and other media components, allowing the ATF to be used for a longer.

In known methods, PES membrane filters (ATF) are typically cleaned with a strong acid and base in combination. The present disclosure provides a method employing very dilute 0.1 to 0.5 N of Sodium Hydroxide (NaOH) alone for inline CIP activity, thereby avoiding weakening of the member allowing it to be used for longer time and reused. Also the use of dilute NaOH avoids damage or degradation of protein products.

While the foregoing describes various embodiments of the disclosure, other and further embodiments of the disclosure may be devised without departing from the basic scope thereof. The invention is not limited to the described embodiments, versions or examples, which are included to enable a person having ordinary skill in the art to make and use the invention when combined with information and knowledge available to the person having ordinary skill in the art.

### EXAMPLES

The disclosure will now be illustrated with working examples, which is intended to illustrate the working of disclosure and not intended to take restrictively to imply any limitations on the scope of the present disclosure. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1

### In Line Cleaning in Process for of an Alternating Tangential Flow (ATF) Filter Implemented in Production of Various Biological Molecules

To cultivate cells in perfusion mode, a Sartorius Biostat BDCUII bioreactor connected to an ATF module with Inline CIP option (Xcell® ATF) was used. CHO-S cells were procured from Catalant, USA. The bioreactor was inoculated at a concentration between 0.5 and 7×10⁶ CHO S cells/mL. The initial production media used was ActiPro media ((Manufacturer: Cytiva) containing 6 mM glutamine. The media was equilibrated for NLT 12 hours before start of the batch. Dissolved oxygen (DO) was controlled in a “cascade” mode through the delivery of both air and oxygen. Airflow was supplied via a rotameter with 0.3 lpm set point and cascade with Oxygen flow. Oxygen was added via a MFC. pH was controlled at 7.0±0.1 by sparging CO2. (On/off—solenoid). A ring sparger was used for sparging air, oxygen & CO². Antifoam C (Sigma) was added manually to reduce foam in bioreactor as and when needed. Perfusion began on day 0 (0 hr) with perfusion media containing ActiPro and cell boost 7a and 7b. Peak cell densities were observed in a range of 60 to 130 million cells/mL and Depending on the VCC, titres from the bioreactor and harvest were seen from days 2 or 3. When the titres were seen to be greater than 60-70% inside the bioreactor, inline CIP was started.

Three to five (apprx-600 mL) hold up volume of NaOH was passed through the ATF hollow fiber filter at flowrate 120-150 ml/L. It was followed by flushing with 10 hold up volumes of autoclaved/filtered WFI. The ATF module was then restarted after being equilibrated with two hold-up volumes of basal or perfusion media.

Different bio molecules are produced and in line CIP process of the present invention in accordance with the present invention was implemented. Summary of different bio-molecules produced is provided in below Table 1.

TABLE 1
Summary of Batch details for different molecules:

| | Therapeutic proteins or Mabs | | | | | | | | | |
|---|---|---|---|---|---|---|---|---|---|---|
| Parameter | Ravulizumab- I | | Ravulizumab - II | | Tocilizumab | | Nivolumab | | Pembrolizumab | |
| Basal Media | Dynamis AGT | | Actipro | | Actipro + 6 mM | | Actipro | | Actipro | |
| | | | | | Gln + 0.5 mM | | | | | |
| | | | | | L-Cysteine + 10 ml/ | | | | | |
| | | | | | L L-Tyrosine | | | | | |
| Batch size (L) | 2 | | 2 | | 2 | | 2 | | 2 | |
| Temperature (° C.) | 37° | C. | 37° | C. | 37° | C. | 37° | C. | 37° | C. |
| Temperature Shift | 34° C. on | | NA | | NA | | 34° C. on | | 34° C. on | |
| (° C.) | Day 5 | | | | | | Day 8 | | Day 11 | |
| pH Set point | 7.00 ± 0.20 | | 7.00 ± 0.20 | | 7.00 ± 0.20 | | 7.00 ± 0.20 | | 7.00 ± 0.20 | |
| Seeding density | 5.34 | | 0.56 | | 0.55 | | 0.57 | | 0.61 | |
| (X10⁶ cells/mL) | | | | | | | | | | |
| Peak VCC | 87.60 - Day 6 | | 76.60 - Day 14 | | 88.80 - Day 11 | | 88.80 - Day 16 | | 75.20 - Day 23 | |
| (X10⁶ cells/mL) | | | | | | | | | | |
| NaOH (Strength) | 0.25N | | 0.15N | | 0.2N | | 0.1N | | 0.15N | |
| NaOH (volume) | 700 | mL | 600 | mL | 500 | mL | 300 | mL | 600 | mL |

Following Tables show effect of CIP implemented on titre values of different bio-molecules mentioned in Table 1:

TABLE 2
Denosumab

| Culture Age(Days) - | Before CIP Titer(g/L) | After CIP Titer(g/L) |
|---|---|---|
| Day 17 Harvest | 0.7 | 0.8 |
| Day 27 Harvest | 0.45 | 0.7 |
| Day 34 Harvest | 0.45 | 0.65 |

TABLE 3
Cetuximab

| Culture Age(Days) - | Before CIP Titer(g/L) | After CIP Titer(g/L) |
|---|---|---|
| Day 12 Harvest | 0.2 | 0.3 |
| Day 14 Harvest | 0.4 | 0.4 |
| Day 16 Harvest | 0.4 | 0.5 |
| Day 18 Harvest | 0.45 | 0.6 |

TABLE 4
Ravulizumab- I

| Culture Age(Days) - | Before CIP Titer(g/L) | After CIP Titer(g/L) |
|---|---|---|
| Day 10 Harvest | 0.45 | 0.57 |
| Day 14 Harvest | 0.37 | 0.45 |

TABLE 5
Ravulizumab - II

| Culture Age(Days) - | Before CIP Titer(g/L) | After CIP Titer(g/L) |
|---|---|---|
| Day 10 Harvest | 0.18 | 0.26 |

TABLE 6
Tocilizumab

| Culture Age(Days) - | Before CIP Titer(g/L) | After CIP Titer(g/L) |
|---|---|---|
| Day 15 | 0.41 | 0.8 |

TABLE 7
Nivolumab

| Culture Age(Days) - | Before CIP Titer(g/L) | After CIP Titer(g/L) |
|---|---|---|
| Day 15 | 1.02 | 1.06 |

TABLE 8
Pembrolizumab

| Culture Age(Days) - | Before CIP Titer(g/L) | After CIP Titer(g/L) |
|---|---|---|
| Day 22 | 1.62 | 1.91 |
| Day 25 | 1.64 | 2.0 |
| Day 26 | 2.0 | 2.46 |

The above Tables show that after CIP, the harvest titre increases, showing the effectiveness of CIP.

This invention has been described in terms of specific embodiments set forth in detail, but it should be understood that these are by way of illustration only and that the invention is not necessarily limited thereto. Modifications and variations will be apparent from this disclosure and may be achieved without departing from the scope of this invention, as those skilled in the art will readily understand. Accordingly, such variations and modifications of the disclosed embodiments are considered to be within the purview and scope of this invention and the following claims.

We claim:

1. A process for an in line cleaning in place (CIP) of an alternating tangential flow (ATF) filter in a filtration module, the filtration module being in communication with a bioreactor, the process comprising the steps of:
i) flowing a dilute NaOH through the ATF filter, the dilute NaOH being 0.1 to 0.5 N NaOH, the volume of NaOH being three to five times the hold up volume of the filter and the flow rate of NaOH being 80-200 ml/L;
ii) flushing the filter with a sterile water, the volume of water being flushed being ten to twenty times the hold up volume of the filter; and
iii) equilibrating the filter with a media.

2. The process as claimed in claim 1, wherein the process includes monitoring a titer of a product in the bioreactor.

3. The process as claimed in claims 1 and 2, wherein the in line CIP is initiated based on the titre value of the accumulated product in the bioreactor.

4. The process as claimed in claim 1, wherein the in line CIP is carried out one or more times based on of titre value of the accumulated product during a phase selected from a log phase, stationary phase and decline phase of growth of cells cultured in the bioreactor.

5. The process as claimed in claim 4, wherein the in line CIP is carried out based on the titre value of the accumulated product in the bioreactor is ≥20% during the lag phase, ≥40% during the log phase, ≥60% during the stationary phase, or <40% and equal to 20% during the decline phase of growth of cells cultured in the bioreactor.

6. The process as claimed in claim 1, wherein the in line CIP is repeated one or more times based on the titre value of the accumulated product in the bioreactor is 60% to 70%.
